(19) United States
(12) Patent Application Publication
(10) Pub. No.: US 2021/0060320 A1
(43) Pub. Date: Mar. 4, 2021

(54) TRANSDERMAL DRUG DELIVERY SYSTEM AND METHOD FOR USING SAME

(71) Applicants: NICHIBAN CO., LTD., Tokyo (JP); SENJU USA, INC., Woodland Hills, CA (US)

(72) Inventors: Teppei OSAKO, Tokyo (JP); Koji KAWAHARA, Tokyo (JP); Takahiro OGAWA, Woodland Hills, CA (US); Akiharu ISOWAKI,, Woodland Hills, CA (US); Tetsuo KIDA, Woodland Hills, CA (US)

(21) Appl. No.: 16/958,071

(22) PCT Filed: Dec. 27, 2018
(86) PCT No.: PCT/US2018/067643

§ 371 Date: Jun. 25, 2020

Related U.S. Application Data
(60) Provisional application No. 62/610,988, filed on Dec. 28, 2017.

(51) Int. Cl. A61M 37/00; A61K 9/00; A61K 31/573; A61F 9/00

(52) U.S. Cl. CPC A61M 37/0015; A61K 9/0021; A61K 9/0048; A61M 2037/0061; A61F 9/0008; A61M 2037/0023; A61K 31/573

(57) ABSTRACT

A method for administering a drug to a diseased portion is achieved in a short time and predominantly improves the administration amount (transdermal permeation amount) of the drug in treatment of eyelid disease or palpebral conjunctiva disease. The method for administering a drug for treatment of eyelid disease or palpebral conjunctiva disease includes a step of microneedle perforation by using a microneedle device into a tissue of the eyelid including a meibomian gland of a patient suffering from or suspected of having the disease, in which drug-containing coating is applied to at least a part of a surface of the microneedle, and the drug is a water-soluble drug.

### TECHNICAL FIELD

The present invention relates to a microneedle device for administering a drug for treatment of eyelid disease or palpebral conjunctiva disease, specifically, a disease such as chalazion, blepharitis, meibomian gland dysfunction, allergic conjunctivitis, and vernal keratoconjunctivitis; and to a method for administering the drug for treatment. The microneedle device for administering a drug for treatment can be regarded as a novel transdermal drug delivery system, and in the present invention, the microneedle device is also referred to as a transdermal drug delivery system.

### BACKGROUND ART

In Patent Document 1, there is a disclosure of a transdermal absorption-type preparation for treatment of eye disease, having a structure in which a plaster layer containing a therapeutic agent for eye disease is provided on a backing film, and there is a description of a steroid patch showing more favorable efficacy and safety than those of an ophthalmic ointment and a method for treatment of eye disease.

Further, in Patent Document 2, there is a disclosure of art for administering a drug by placing a drug-containing hydrogel on a surface of the skin after perforation by microneedle array treatment with a needle length of several hundred microns, as a simple and compact transdermal drug delivery device, and there is a description that the drug permeation amount can be significantly improved by controlling the shape retainability of the drug-containing hydrogel within a predetermined range.

### PRIOR ART DOCUMENT

### Patent Document

Patent Document 1: Japanese National Publication 2014-519955 (SENJU USA)
Patent Document 2: Japanese Patent No. 5767094 (Nichiban)

### Non-Patent Document

Non Patent Document 1: Journal of American Academy of Dermatology 2006; 54: 1-15
Non Patent Document 2: Survey of Ophthalmology 1979; 24: 57-88.
Non Patent Document 3: British Journal of Dermatology 1976; 95: 207-208
Non Patent Document 4: Archives of Dermatology 1976; 112: 1326
Non Patent Document 5: Archives of Dermatology 1978; 114: 953-954
Non Patent Document 6: Ophthalmology 1997; 104: 2112-2116

### SUMMARY OF THE INVENTION

### Problems to be Solved by the Invention

The art disclosed in the above-described Patent Document 1, that is, a means for directly applying a transdermal absorption-type preparation to the eyelid skin gives restraint on daily activities, so that the patient feels certain burdens.

Further, in the art disclosed in Patent Document 2, since a drug-containing hydrogel is placed on a surface of the eyelid skin, the influence on the quality of life (QOL) of the patient is concerned. In addition, in the same Patent Document 2, there is no specific description about the effective treatment of diseases of the eyelid and the palpebral conjunctiva, such as chalazion, blepharitis, meibomian gland dysfunction, allergic conjunctivitis, and vernal keratoconjunctivitis.

An object of the present invention is to provide a method for administering a drug to a disease portion in a short time and advantageously improving the administration amount (transdermal permeation amount) of the drug, in treatment of eyelid disease or palpebral conjunctiva disease, in particular, a disease of the eyelid or the palpebral conjunctiva, such as chalazion, blepharitis, meibomian gland dysfunction, allergic conjunctivitis, and vernal keratoconjunctivitis.

### Means to Solve the Problems

The present inventors have found that as a novel transdermal drug delivery system, a drug can be administered to a diseased portion of the eyelid or the palpebral conjunctiva in a short time and with an effective therapeutic concentration by constituting a microneedle device in which a surface of a microneedle is coated with a water-soluble drug for treatment of eyelid disease or palpebral conjunctiva disease and by conducting the microneedle perforation into a tissue of the eyelid including a meibomian gland. The present inventors thus completed the present invention.

That is, the present invention relates to:
[1] A method for administering a drug for treatment of eyelid disease or palpebral conjunctiva disease, including a step of conducting a microneedle perforation by using a microneedle device into a tissue of the eyelid including a meibomian gland of a patient that has been infected with or that may be infected with the disease, in which drug-containing coating is applied to at least a part of a surface of the microneedle, and the drug is a water-soluble drug.

According to the present invention, the following embodiment is further provided.
[2] The method described above, in which the drug is at least a water-soluble steroid having an octanol/water distribution coefficient (log D) selectable in a range from −5 to 0.
[3] The method described above, in which the drug is at least one water-soluble steroid selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.
[4] The method described above, in which the drug-containing coating contains at least one kind of water-soluble polymer selectable from the group consisting of polyhydroxymethyl cellulose, polyhydroxypropyl methyl cellulose, polymethyl cellulose, hydroxyethyl cellulose, hydroxypropyl cellulose, hydroxypropyl methyl cellulose, carboxymethyl cellulose, carboxypropyl cellulose, dextran, polyethylene glycol, polyvinyl alcohol, polyvinyl pyrrolidone, pullulan, chondroitin sulfate or an ester salt thereof, and hyaluronic acid or a salt thereof.
[5] The method described above, in which the step of conducting a microneedle perforation is a step of conducting a microneedle perforation into a surface of the eyelid skin or a surface of the palpebral conjunctiva.
[6] The method described above, in which the step of conducting the microneedle perforation is a step of conducting a microneedle perforation for 1 second to 10 seconds.
[7] The method described above, further including a step of applying a vasoconstrictor to the tissue of the eyelid.
[8] The method described above, in which the eyelid disease or palpebral conjunctiva disease is at least one disease selectable from the group consisting of chalazion, blepharitis, meibomian gland dysfunction, allergic conjunctivitis, and vernal keratoconjunctivitis.

Further, the present invention relates to:
[9] A microneedle device for administering a drug for treatment of eyelid disease or palpebral conjunctiva disease to a tissue of the eyelid including a meibomian gland, including a microneedle, in which coating containing the drug is applied to at least a part of a surface of the microneedle, and the drug is a water-soluble drug.

According to the present invention, the following embodiment is further provided.
[10] The microneedle device described above, in which the microneedle device is a device for administering the drug through the eyelid skin or the palpebral conjunctiva.
[11] A microneedle device for administering a drug for treatment of eyelid disease or palpebral conjunctiva disease to a tissue of the eyelid including a meibomian gland through the eyelid skin or the palpebral conjunctiva, including a microneedle, in which coating containing the drug is applied to at least a part of a surface of the microneedle, and the drug is a water-soluble drug.
[12] The microneedle device described above, in which the drug is at least a water-soluble steroid having an octanol/water distribution coefficient (log D) selectable in a range from −5 to 0.
[13] The microneedle device described above, in which the drug is at least one water-soluble steroid selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.
[14] The microneedle device described above, in which the coating contains at least one kind of water-soluble polymer selectable from the group consisting of polyhydroxymethyl cellulose, polyhydroxypropyl methyl cellulose, polymethyl cellulose, hydroxyethyl cellulose, hydroxypropyl cellulose, hydroxypropyl methyl cellulose, carboxymethyl cellulose, carboxypropyl cellulose, dextran, polyethylene glycol, polyvinyl alcohol, polyvinyl pyrrolidone, pullulan, chondroitin sulfate or an ester salt thereof, and hyaluronic acid or a salt thereof.
[15] The microneedle device described above, in which the eyelid disease or the palpebral conjunctiva disease is at least one disease selectable from the group consisting of chalazion, blepharitis, meibomian gland dysfunction, allergic conjunctivitis, and vernal keratoconjunctivitis.
[16] The microneedle device described above, in which the microneedle device is used for treatment of eye disease due to dysfunction of a tissue of the eyelid.
[17] The microneedle device described above, in which the microneedle device is used for treatment of eye disease.
[18] The microneedle device described above, further including a microneedle array provided with the microneedle, in which the microneedle array has an area of 0.01 cm² or more and 1.20 cm² or less.
[19] A method for treatment of eyelid disease or palpebral conjunctiva disease, including a step of making a microneedle in the microneedle device described above perforated into the eyelid skin or a surface of the palpebral conjunctiva of a patient that has been infected with or that may be infected with disease.
[20] The method described above, in which the step of conducting the microneedle perforation is a step of conducting a microneedle perforation for 1 second to 10 seconds.
[21] The method described above, further including a step of applying a vasoconstrictor to a tissue of the eyelid.
[22] A set for treatment of eyelid disease or palpebral conjunctiva disease, including the microneedle device described above, and a drug product containing one or both of a support base for eyelid skin and a vasoconstrictor.

### Effect of the Invention

According to the present invention, in treatment of eyelid disease or palpebral conjunctiva disease, in particular, treatment of a disease such as chalazion, blepharitis, meibomian gland dysfunction, allergic conjunctivitis, and vernal keratoconjunctivitis, the administration of a drug to a diseased portion in a short time with an effective therapeutic concentration can be realized.

In particular, in the present invention, without requiring any treatment or the like, for example, treatment with an adhesive skin patch or the like over several hours in a diseased portion, which has been required so far, the administration of a drug to a diseased portion is completed in a short time (several seconds) by conducting a microneedle perforation, on a surface of which a water-soluble drug has been coated, into a diseased portion or the surrounding tissue thereof. Further, this treatment in a short time reduces the number of administration steps, reduces the uncomfortable feeling (foreign body sensation) in a diseased portion, which is accompanied by the conventional treatment (application of an adhesive skin patch), and reduces the burden on a patient without giving restraint on the daily activities of the patient.

Further, in the present invention, by using a microneedle on which a drug has been directly carried, the specific drug administration from a surface of the skin (mucosal) at a diseased portion can be realized by conducting the microneedle perforation into the target site.

In addition, by adjusting the amount of the drug to be carried on the microneedle, it is easy to adjust the drug dose. That is, there is an advantage that the drug dose can be adjusted depending on the severity, further the frequency of administration can be adjusted, and the like.

### EMBODIMENTS CARRYING OUT THE PRESENT INVENTION

The present invention relates to a method for administering a drug for treatment of eyelid disease or palpebral conjunctiva disease, and to a microneedle device for administering a drug for treatment of eyelid disease or palpebral conjunctiva disease.

Specifically, the above-described method is performed for a patient (subject of, for example, human, rabbit, dog, cat, cow, horse, monkey or the like) that has been infected with or that may be infected with ophthalmic diseases or the palpebral conjunctiva, such as chalazion, blepharitis, meibomian gland dysfunction, allergic conjunctivitis, and vernal keratoconjunctivitis, with the use of a microneedle device, by conducting perforation of a microneedle, at least a part of a surface of which has drug-containing coating, into a tissue of the eyelid including a meibomian gland of the patient. More specifically, the method is performed by being perforated into a surface of the eyelid skin or a surface of the palpebral conjunctiva of the patient.

The eyelid skin is a surface of the skin including a surface of the eyelid (lid of the eye) and refers to the front of the upper eyelid, the lower eyelid or both eyelids, or surfaces of the skins of these eyelids. Further, the palpebral conjunctiva refers to a membrane on the back side of the eyelid, and refers to the upper palpebral conjunctiva, the lower palpebral conjunctiva, or conjunctivae of both eyelids.

Hereinafter, first, the microneedle device that is used in the method according to the present invention method, and is also an object of the present invention, that is, a microneedle device provided with a microneedle to which drug-containing coating has been applied will be described in detail.

### [Microneedle Device]

The microneedle device to be used in the present invention is provided with a microneedle, on at least a part of a surface of which has drug-containing coating. Further, the above-described microneedle device is used for administration of a drug by making the above-described microneedle perforated into a surface of the eyelid skin or a surface of the palpebral conjunctiva. In this regard, administration can be performed from either the eyelid skin side or the palpebral conjunctiva side, but it is preferred that the administration is performed from the palpebral conjunctiva side because a large amount of a drug can be transferred to the meibomian glands. This is presumed because the distance to the meibomian glands from the palpebral conjunctiva side is closer than that from the eyelid skin side. In addition, when the administration (perforation) is performed from the eyelid skin side, since the perforated portion is exposed (the perforated portion is a surface of the face and can be visually observed), the place where the perforation has been performed becomes noticeable by the color of a liquid medicine in a case where the liquid medicine is colored. Further, even if the liquid medicine is colorless, a scratch (perforation hole) generated when the perforation is performed becomes noticeable depending on the shape of a microneedle, and therefore, the administration (perforation) from the eyelid skin side is not preferred.

In this regard, in general, in a microneedle device, microneedles are provided in a form of a microneedle array in which usually two or more microneedles are arranged longitudinally and transversely, and the treatment of making the microneedle array perforated into the skin or the like is also referred to as a microneedle array treatment.

Further, in the present invention, the microneedle device may be a form of including a microneedle array that is provided with microneedles.

That is, the method according to the present invention can also be referred to as a method in which a microneedle array treatment is performed on a surface of the eyelid skin or a surface of the palpebral conjunctiva, and at the same time, a drug that has been coated on the microneedles constituting the microneedle array is administered.

The specific method and form of the above-described microneedle array treatment are not particularly limited, and for example, the microneedle array treatment can be performed by any instrument capable of temporarily reducing the barrier function of the eyelid skin by conducting perforation of multiple needles at the same time (see, for example, Wu, X. M., et al., (2006) J. Control Release, 118: 189-195, and the like).

The constituent materials of the above-described microneedle are not particularly limited, and for example, a synthetic plastic microneedle having a substrate made of polycarbonate, polyurethane, polymethacrylate, ethylene-vinyl acetate copolymer, polytetrafluoroethylene, polyoxymethylene, polyester, nylon, polystyrene, or polyolefin, an autolysis microneedle having a substrate made of polylactic acid, polycaprolactone, or polyglycolic acid, or a microneedle made of silicon (compound), silicon dioxide, ceramic, or a metal (stainless steel, iron, aluminum, titanium, nickel, or the like) can be used.

The shape and size of a microneedle are also not particularly limited, and a microneedle usually has a cone shape such as a conical shape or a polygonal pyramid shape (triangular pyramid shape, quadrangular pyramid shape, or the like), and, for example, in a case of having a triangular pyramid shape, the area of the bottom can be set to around 0.1 mm² to 0.5 mm², the height of the pyramidal shape can be set to around 0.2 mm to 0 5 mm, and the diameter of the pyramidal tip can be set to around 1 μm to 30 μm. When the height of the pyramidal shape is less than 0.2 mm, there is a risk that a drug cannot be sufficiently transferred to the meibomian glands because the skin cannot be scratched to have a sufficient depth, and therefore, this is not preferred. When the height of the pyramidal shape exceeds 0.5 mm, pain is generated, and a considerable amount of bleeding will occur, and therefore, this is not preferred. Further, when the diameter of the pyramidal tip is less than 1 μm, since the tip strength of a needle cannot be kept and the needle is broken at the time of perforation, a drug cannot be transferred to the meibomian glands, and therefore, this is not preferred. When the diameter of the pyramidal tip exceeds 30 μm, the needle does not get stuck at the time of perforation and a drug cannot be transferred to the meibomian glands, and therefore, this is not preferred.

In addition, the size of a microneedle array is not particularly limited for the application area, and may be set to, for example, an area equal to, smaller than, or larger than the application area of the method. The size of a microneedle array may be preferably set to an area equal to or smaller than the application area of the method. For example, in a case where the application to the eyelid skin or the palpebral conjunctiva is assumed, the area of the microneedle array is suitably 0.01 cm² or more and 1.20 cm² or less. When the area of the microneedle array is less than 0.01 cm², a therapeutically effective amount of the drug to be transferred cannot be expected. When the area of the microneedle array is 1.20 cm² or more, the uncomfortable feeling accompanying the increase of the area is increased at the time of perforation, while there is no difference in the amount of the drug to be transferred to the meibomian glands, and therefore, this is not preferred.

In addition, the set number (the number of needles) of the microneedles constituting a microneedle array can be appropriately set, and for example, can be set to 1 needle to 500 needles. The density of the microneedles can be set to 0.8 needle/cm² to 1000 needles/cm². When the density is less than 0.8 needles/cm², a sufficient amount of liquid medicine cannot be carried on a microneedle device, and therefore, a sufficient amount of a drug cannot be transferred to the meibomian glands. When the density exceeds 1000 needles/cm², the microneedles hardly stick the skin because the force applied to one microneedle at the time of perforation is dispersed, and therefore, this is not preferred.

As described above, by changing the shape and size (the area of the bottom, the height of the pyramidal shape, and the like) of the microneedle, the size of the microneedle array, and the set number of the microneedles, the coating amount of the drug to be applied to the whole microneedles provided in a microneedle device is adjusted, and thus the drug dose to a diseased portion can be adjusted.

### [Drug-Containing Coating]

The microneedle to be used in the present invention is obtained with a coating that has a drug on a part of a surface of a microneedle.

The coating may contain a water-soluble drug as a drug, preferably a carrier such as a water-soluble polymer, and other additives described later as needed.

### <Water-Soluble Drug>

The water-soluble drug to be used in the present invention is not particularly limited, and a growth factor, a hormone, a cytokine, an antigen, an antibody, a fragment and an analog thereof, and the like in addition to a common water-soluble drug can be mentioned as a preferred drug as long as they are water soluble. Among them, as a suitable water-soluble drug, a water-soluble steroid that is expected to have a high curing effect and an immediate effect on inflammatory eyelid disease or palpebral conjunctiva disease can be mentioned.

As the water-soluble steroid to be used in the present invention, an arbitrary pharmacologically acceptable water-soluble steroid can be mentioned, and examples of the pharmacologically acceptable water-soluble steroid include dexamethasones such as dexamethasone sodium phosphate, and dexamethasone metasulfobenzoate sodium; hydrocortisones such as hydrocortisone sodium phosphate, and hydrocortisone sodium succinate; prednisolones such as prednisolone sodium phosphate, and prednisolone sodium succinate; methylprednisolones such as methylprednisolone sodium succinate; and betamethasones such as betamethasone sodium phosphate. Among them, dexamethasone sodium phosphate can be mentioned as a suitable drug.

As other water-soluble drugs that can be used in the present invention, diquafosol sodium, and lifitegrast can be mentioned, and further a non-steroidal anti-inflammatory drug such as diclofenac sodium, bromfenac sodium, pranoprofen, and ketorolac tromethamine; an antibiotic such as cefmenoxime hydrochloride, ofloxacin, norfloxacin, lomefloxacin hydrochloride, levofloxacin, tosufloxacin tosilate, gatifloxacin, moxifloxacin hydrochloride, gentamicin sulfate, tobramycin, dibekacin sulfate, fradiomycin sulfate, chloramphenicol, colistin, vancomycin hydrochloride, erythromycin, azithromycin, clarithromycin, and doxycycline; an immunosuppressive agent such as ciclosporin, and tacrolimus; an anti-allergic agent such as sodium cromoglicate, ketotifen fumarate, pemirolast potassium, tranilast, ibudilast, acitazanolast, levocabastine hydrochloride, olopatadine hydrochloride, epinastine hydrochloride, and bepotastine besilate; a biologically active substance such as a heparinoid; peptides such as substance P; a polysaccharide such as maltooligosaccharide; a cyclarean extract; an extract derived from a natural product having a sebum secretion promoting action, which contains at least one kind of extract selected from the group consisting of cnidium rhizome, Sophora flavescens, Tussilago farfara, Tilia cordata, Poria sclerotium, and Lavandula angustifolia; and the like can be mentioned.

Note that in the present invention, as an index of the “water solubility” in a “water-soluble drug” or a “water-soluble steroid”, a distribution coefficient (octanol/water distribution coefficient) can be employed. The distribution coefficient can be expressed by log D by taking into consideration of the influence of pH, and the water solubility is indicated by a minus value, while the liposolubility lipophilicity is indicated by a plus value.

The range of the distribution coefficient (log D) of the compound to be used is not particularly limited, and from the viewpoint of the skin permeability, it is preferred to use a compound having a distribution coefficient (log D) in a range of from −5 to 0 at pH 8.

For example, as an example of the distribution coefficient (log D) at pH 8 in the above-described compound as the water-soluble steroid, dexamethasone sodium phosphate: −5, dexamethasone metasulfobenzoate sodium: −2, prednisolone sodium phosphate: 0, or betamethasone sodium phosphate: −5 can be mentioned.

The distribution coefficient can be obtained in accordance with the method described in JIS Z7260-107 or JIS Z6260-117.

The blending amount and dose of the above-described water-soluble drug (water-soluble steroid) vary depending on the kind of the drug, the symptoms of the subject, and the age and body weight of the subject.

In general, the water-soluble drug (water-soluble steroid) is administered 1 mg to 15 mg per dose and 2 mg to 30 mg per day. In a case where the dosage is 1 mg/dose and less than 2 mg/day, there is no effect on the treatment of eyelid disease or palpebral conjunctiva disease, and in a case where the dosage is 15 mg/dose and exceeds 30 mg/day, side effects (shock, anaphylactic reaction, or the like) appear, and therefore, this is not preferred. Note that the indication of this dosage also applies to the therapeutic agent used for the eye disease as described later.

### <Carrier: Water-Soluble Polymer>

The drug-containing coating preferably contains a carrier in order to apply (carry) the water-soluble drug to a microneedle. As such a carrier, a carrier that keeps a drug so as to prevent the drug from dropping off from a microneedle before the administration (perforation into a diseased portion), and further is desorbed into the perforation portion together with the drug immediately after the administration (perforation), and releases the drug to the perforation hole or the surrounding tissue thereof by the dissolution or the like is preferred.

As such a carrier, a water-soluble polymer can be preferably mentioned, and examples of the water-soluble polymer include polyhydroxymethyl cellulose, polyhydroxypropyl methyl cellulose, polymethyl cellulose, hydroxyethyl cellulose, hydroxypropyl cellulose, hydroxypropyl methyl cellulose, carboxymethyl cellulose, carboxypropyl cellulose, dextran, polyethylene glycol, polyvinyl alcohol, polyvinyl pyrrolidone, carrageenan, xanthane gum, pullulan, chondroitin sulfate or an ester salt thereof, and hyaluronic acid or a salt thereof.

Among them, as a water-soluble polymer being a suitable carrier, hydroxypropyl cellulose can be mentioned, and for example, a commercially available product such as NISSO HPC SSL (weight-average molecular weight in terms of pullulan by a gel permeation chromatography (GPC) method: up to 40,000), NISSO HPC SL (weight-average molecular weight in terms of pullulan by a GPC method: up to 100,000), NISSO HPC L (weight-average molecular weight in terms of pullulan by a GPC method: up to 140,000), NISSO HPC LM (weight-average molecular weight in terms of pullulan by a GPC method: up to 180,000), and NISSO HPC LMM (weight-average molecular weight in terms of pullulan by a GPC method: up to 280,000) all manufactured by Nippon Soda Co., Ltd. can be suitably used. As the molecular weight (molecular weight by a GPC method), it is preferably 40,000 to 280,000. When the molecular weight is less than 40,000, the uniform coating cannot be applied, and when the molecular weight exceeds 280,000, the drug migration is not sufficient, and therefore, these cases are not preferred.

### <Method for Applying Drug-Containing Coating to Microneedle>

In the method for applying a drug-containing coating to a microneedle, a liquid medicine (coating liquid) in a liquid state or suspension state is prepared by blending the above-described water-soluble drug (water-soluble steroid), a water-soluble polymer, other additives as needed, and a volatile liquid as a solvent, and then the prepared liquid medicine may be applied to a microneedle and dried. For example, by immersing a microneedle in a coating liquid containing the above-described water-soluble drug and the like, pulling the immersed microneedle up, and drying the pulled-up microneedle, for example, if the microneedle has a cone shape, a coating containing the water-soluble drug is allowed to adhere onto the outer peripheral surface of the cone, and the drug can be carried. At this time, the immersion and the drying may be repeated so as to have a desired amount of the water-soluble drug to be carried.

The blending amount of the above-described water-soluble drug (water-soluble steroid) in the coating liquid described above can be determined in consideration of the above-mentioned dosage, and is preferably blended so that the water-soluble drug has a concentration of, for example, 5% by mass to 20% by mass relative to the total mass of the coating liquid. In a case where the mixing amount is less than 5% by mass, there is no effect on the treatment of eyelid disease or palpebral conjunctiva disease, and in a case where the blending amount exceeds 20% by mass, side effects (shock, anaphylactic reaction, and the like) appear, and therefore, this is not preferred.

In addition, the blending amount of the water-soluble polymer being a carrier is preferably, for example, 10% by mass to 50% by mass. When the mixing amount is less than 10% by mass, the uniform coating cannot be applied, and when the mixing amount exceeds 50% by mass, the drug migration is not sufficient, and therefore, these cases are not preferred.

As the volatility liquid, water, a saline solution, dimethyl sulfoxide, dimethylformamide, ethanol, isopropyl alcohol, or a mixture thereof can be used. Among them, water, or a saline solution is most preferred. The volatility liquid is preferably blended so as to have a concentration of, for example, 20% by mass to 90% by mass relative to the total mass of the coating liquid.

In the above-described coating liquid (liquid medicine), a stabilizer, a plasticizer, a buffer agent, a base, a suspending agent, an antioxidant, a coloring agent, an emulsifier, a thickening agent, a pH regulator, a dispersant, an antiseptic agent, a preservative, a solvent, a surfactant (a nonionic surfactant, a cationic surfactant, an anionic surfactant, or an amphoteric surfactant), salts, and the like may be added.

In addition, in the above-described coating liquid, as a medicinal aid, a transdermal absorption promoting agent, or a dissolution aid of a drug, for example, crotamiton, L-menthol, mentha oil, limonene, diisopropyl adipate, methyl salicylate, glycol salicylate, thymol, mentha oil, nonylic acid vanillylamide, capsicum extract, and the like may be blended.

When these other additives, other known preparation auxiliary substances, and the like are used, the kind and the blending amount can be appropriately determined as long as they do not adversely affect the necessary solubility and viscosity, the physical properties (shape retainability) of the coating after drying and coating, and further, the skin irritation due to the applied coating, the skin permeability of the water-soluble drug, and the like in the application of the coating liquid to a microneedle.

In the microneedle device according to the present invention, the coating applied to the above-described microneedle, for example, contains water as the solvent (volatility liquid), includes a combination of a water-soluble steroid (water-soluble drug) such as dexamethasone sodium phosphate and a water-soluble polymer (carrier) such as hydroxypropyl cellulose, and has an unprecedented coating composition.

Further, by not using a non-volatility component such as glycerin as a solvent or a carrier, this coating becomes a coating having an adequate hardness by drying, and therefore, the release of the drug (coating) at the time of the microneedle perforation can be prevented. On the other hand, in a case where an oily substance such as glycerin is blended into the coating, the applied coating may become extremely soft, the coating (drug) may be come off at the time of the microneedle perforation, that is, so to say, only the microneedle may be introduced, and as a result of which the drug may not be released to the perforation hole and the like in some cases.

In addition, in the present invention, since a component that dissolves well in water, such as a water-soluble polymer is used as the coating composition, when the microneedle is perforated into the eyelid skin, in particular, the palpebral conjunctiva, the water-soluble polymer and the like in the coating are rapidly dissolved due to the intradermal moisture, the drug is released from the coating and desorbed from the microneedle, and can be transferred into the skin This is considered to advantageously work on the administration time of the drug.

### [Method for Administering Drug for Treatment of Eyelid Disease or Palpebral Conjunctiva Disease]

As described above, the method for administering a drug for treatment of eyelid disease or palpebral conjunctiva disease according to the present invention includes a step of conducting a microneedle perforation using the above-described microneedle device into a tissue of the eyelid including a meibomian gland of a patient suffering from or suspected of having the disease.

The above-described step of conducting the microneedle perforation is preferably a step of conducting a microneedle perforation into a surface of the eyelid skin or a surface of the palpebral conjunctiva, and particularly preferably a step of conducting a microneedle perforation into a surface of the palpebral conjunctiva.

The step of conducting a microneedle perforation is suitably a step of conducting a microneedle perforation for 1 second to 10 seconds, for example, 1 second, 3 seconds, 5 seconds, or 7 seconds. By arranging the step of conducting a microneedle perforation longer, the dissolution of a component such as a water-soluble polymer in the coating proceeds due to the intradermal moisture in the eyelid skin or the palpebral conjunctiva, the release and desorption of the drug from a microneedle proceed, and the transfer of the drug into the skin is promoted. However, even if this step is arranged much longer, no further effect cannot be obtained after the coating is completely dissolved, and there is a fear that the failure (bleeding and tissue damage) of the tissue to be subjected to perforation is increased.

The above-described method according to the present invention may include a step of applying a vasoconstrictor to the tissue of the eyelid. The step of applying a vasoconstrictor may be performed before the step of the microneedle perforation, during the step of the microneedle perforation, or after the step of the microneedle perforation. In addition, from the viewpoint of efficiently preventing the bleeding at a perforation portion of a microneedle, the step of applying a vasoconstrictor is preferably performed before the perforation or during the perforation, and further, from the viewpoint of the simplicity of perforation operation, the step of applying a vasoconstrictor is most preferably performed before the perforation.

As the above-described vasoconstrictor, a common vasoconstrictor that can be applied to the eyelid, the palpebral conjunctiva, and the entire eye part including the eyelid and the palpebral conjunctiva may be used, and further can be applied in an amount normally applied to these tissues. Examples of the vasoconstrictor include an al receptor agonist such as naphazoline hydrochloride, and oxymetazoline hydrochloride, tetrahydrozoline hydrochloride, midodrine hydrochloride, sumatriptan succinate, and zolmitriptan.

### [Method for Treatment of Eyelid Disease or Palpebral Conjunctiva Disease]

In addition, an object of the present invention is also a method for treatment of eyelid disease or palpebral conjunctiva disease, and the method includes a step of conducting a microneedle perforation in the above-described microneedle above into a surface of the eyelid skin or the palpebral conjunctiva of a patient suffering from or suspected of having disease.

The above-described step of conducting the microneedle perforation can be a step of conducting a microneedle perforation for 1 second to 10 seconds, for example, 1 second, 3 seconds, 5 seconds, or 7 seconds similarly as in the <step of conducting a microneedle perforation> in the above [method for administering drug for treatment of eyelid disease or palpebral conjunctiva disease].

Further, the above-described treatment method may include a step of applying a vasoconstrictor to the tissue of the eyelid. As the vasoconstrictor, the ones described above can be mentioned, and the step of applying a vasoconstrictor may be performed before the step of the microneedle perforation, during the step of the microneedle perforation, or after the step of the microneedle perforation. In this regard, as described above, from the viewpoint of efficiently preventing the bleeding at a perforation portion of a microneedle, the vasoconstrictor is preferably administered before the perforation or during the perforation, and further, from the viewpoint of the simplicity of perforation operation, the vasoconstrictor is most preferably administered before the perforation.

Conventionally, as the conventional method for administering a drug for a disease of the eye part, particularly the eyelid skin, or the palpebral conjunctiva, the use of eye drops, ointment, or an adhesive skin patch such as a patch, the direct administration of a drug by injection, or the like is performed.

Ointment and eye drops cannot be said to have favorable skin permeability depending on the drug, and may cause side effects due to the long-term use, and the administration in a form of patch gives significant uncomfortable feeling (foreign body sensation) during the administration. Further, in a case where a drug is directly administered (injected) to the eye part, for example, under the conjunctiva or under the Tenon capsule, not only it is difficult for a non-medical professional to perform the administration, but also the psychological burden on a medical professional and a patient is not small, with associated risks of the infection with bacteria or the like and of the damage to a tissue of the eye part.

On the other hand, the microneedle device provided with the microneedle to which a coating of a water-soluble drug has been applied, according to the present invention can realize the drug administration specific to a target site of a meibomian gland or the like as compared with instillation administration, ointment administration, and an adhesive skin patch, and further as compared with systemic administration (oral, intravenous, subcutaneous, intramuscular, or the like). Therefore, by using the microneedle device according to the present invention, the drug migration to the target site becomes rapid, and the drug in a necessary and sufficient amount can be efficiently administered. Further, the microneedle device according to the present invention can reduce the drug exposure on a site other than the target site as compared with the systemic administration, and is greatly advantageous in terms of the adherence, the safety, and the like. Furthermore, the drug administration in a short time can be realized, and therefore, the uncomfortable feeling (foreign body sensation) during the administration is reduced.

In addition, the drug administration by the microneedle device according to the present invention reduces the risks of the infection with bacteria or the like and of the damage to the eye part, as compared with the ocular topical administration such as the direct administration under the conjunctiva, the injection under the Tenon capsule, or the intraocular injection, and therefore, the psychological burden on a medical professional and further on a patient is small.

As described above, as compared with the conventional treatment method for a disease of the eye part, particularly the eyelid skin or the palpebral conjunctiva, the microneedle device according to the present invention is dominant in the application rate (rapid administration) and the administration concentration (sufficient concentration), and thus can contribute to the treatment and improvement of the diseased portion in a short period of time. From the viewpoint of reducing the burden at the time of treatment or of suppressing the side effects due to long-term use, and also from the viewpoint of the improvement of adherence, the safety or the like, the microneedle device is expected to be a useful transdermal drug delivery system.

Further, the microneedle device according to the present invention can also be expected to contribute to the drug migration to other tissues of the eye through a meibomian gland, and is expected to be a dominant transdermal drug delivery system in treatment of not only eyelid disease or palpebral conjunctiva disease but also eye disease due to dysfunction of a tissue of the eyelid typified by the dry eye caused by abnormal lipid release from a meibomian gland, and further in the whole treatment of an eye disease such as cataract, glaucoma, chalazion, conjunctivitis, infection, corneal endothelial disorder, uveitis, endophthalmitis, retinopathy, age-related macular degeneration, and dry eye.

### [Set of Treating for Eyelid Disease or Palpebral Conjunctiva Disease]

The above-described microneedle device according to the present invention can be used as a set of treating for eyelid disease or palpebral conjunctiva disease together with a drug product containing a support base for eyelid skin and/or a vasoconstrictor. The above-described set for treatment of eyelid disease or palpebral conjunctiva disease is also an object of the present invention.

In this set, as a microneedle device, the microneedle device according to the present invention provided with a microneedle, on at least a part of a surface of which drug-containing coating has been applied, or also the microneedle device according to the present invention, which is in a form of containing a microneedle array provided with the microneedle can be used. By adopting the form of such a set, even in the administration method on the eyelid skin side or the palpebral conjunctiva side, or other administration methods, the administration can be performed without touching the inside of the eye part directly with the hand, and therefore, infections can be prevented, and this is advantageous from the viewpoint of the hygiene.

### <Support Base for Eyelid Skin>

The skin that is rich in flexibility, such as the eyelid, is not sufficiently perforated simply by the conventional microneedle perforation treatment (microneedle array treatment), and there are not a few problems that the effect of improving the drug permeation cannot be obtained. On the other hand, by providing certain stiffness to the skin to be perforated, sufficient perforation of the microneedle will become possible. As a method for providing the certain stiffness to the skin to be perforated, a base for supporting the corresponding skin may be used.

That is, the above-described support base for eyelid skin is referred to as a base to support the eyelid, which is used by being inserted into, for example, a gap between the eyelid skin and the eyeball when perforation treatment of the eyelid skin is performed with the microneedle (microneedle array).

The above-described support base for eyelid skin is, for example, placed so as to sandwich the eyelid from the front side (skin) and the back side (conjunctiva) by inserting a part of the base into the gap between the eyelid skin and the eyeball, and to invert and expose the palpebral conjunctiva. Alternatively, the above-described support base for eyelid skin is placed so as to sandwich the eyelid skin with a microneedle (or a microneedle array) by being inserted from the conjunctiva side on the inside of the eyelid into the gap between the eyelid skin and the eyeball at the time of performing microneedle perforation treatment (microneedle array treatment).

As the above-described support base for eyelid skin, it is not particularly limited, and any support base for eyelid skin can be used as long as it has appropriate size and thickness so that at least a part of the support base for eyelid skin is inserted between the eyelid skin and the eyeball, and has the function described above. For example, entropion forceps, or a corneal protection plate (lid plate, also called tapetum) can be used as the support base for eyelid skin. A corneal protection plate (lid plate) means one of the medical instruments commonly used in an ophthalmological clinic.

When the perforation is performed to the palpebral conjunctiva by using the entropion forceps, a frame (window part) of the entropion forceps is inserted from the conjunctiva side on the inside of the eyelid, a plate part (plate-shaped component) of the entropion forceps is placed on the eyelid skin, the eyelid skin (and the palpebral conjunctiva) is sandwiched by the frame and the plate part of the entropion forceps to stretch the skin and to apply tension to the skin, further the skin is inverted to expose the palpebral conjunctiva, and the palpebral conjunctiva in the frame (window part) of the entropion forceps may be subjected to perforation treatment with a microneedle. Alternatively, conversely, in a case of conducting perforation into the eyelid skin, the plate part (plate-shaped component) of the entropion forceps is inserted into the conjunctiva side on the inside of the eyelid, which is the side opposite to the side of the skin to be perforated, and sandwiched the eyelid skin with the frame (window part) of the entropion forceps from the side of the skin to be perforated to stretch the skin and to apply tension to the skin, and then the eyelid skin in the frame (window part) of the entropion forceps may be subjected to perforation treatment with a microneedle.

Further, in a case of using a corneal protection plate (lid plate), the contact surface on the eyelid side of the corneal protection plate (lid plate) is inserted into the conjunctiva side on the inside of the eyelid, which is the side opposite to the side of the skin to be perforated, and the eyelid skin held by the corneal protection plate (lid plate) may be subjected to perforation treatment with a microneedle.

In addition, a function (component) that serves as the above-described support base for eyelid skin is provided to a microneedle array, and as a form of the “microneedle device” having a function of the support base for eyelid skin, a form of having a function of sandwiching and perforating into the eyelid skin or the palpebral conjunctiva may be provided.

Further, as the drug product containing a vasoconstrictor, which may be included in the above-described set of treating for eyelid disease or palpebral conjunctiva disease, the drug products described above can be adopted.

### EXAMPLES

Hereinafter, the present invention will be described in more detail by way of Examples. These Formulation Examples and Examples are merely illustrative, and are not intended to limit the scope of the present invention.

Note that in the present Examples, the expression “%” means “% by mass” in the composition ratio of a mixture.

### <<Microneedle Device (Microneedle Array)>>

A microneedle array (diameter: 8 mm) having 305 microneedles per array, in which the microneedles made of polycarbonate have a conical shape (height: 300 μm×bottom diameter: 300 μm), was used.

### Production Example

### Preparation of Coating Liquid

In accordance with the following procedures, a coating liquid for coating a microneedle was prepared.

TABLE 1

| Prescrip- | | Trade | Manufacturing | Blended |
|---|---|---|---|---|
| tion | Component | name | company | ratio (%) |
| A | Hydroxypropyl | NISSO | Nippon Soda | 15 |
| | cellulose | HPC L | Co., Ltd. | |
| | Dexamethasone | — | Crystal | 10 |
| | sodium phosphate | | Pharma S.a.u. | |
| | Water | — | — | 75 |
| B | Hydroxypropyl | NISSO | Nippon Soda | 30 |
| | cellulose | HPC SSL | Co., Ltd. | |
| | Dexamethasone | — | Crystal | 10 |
| | sodium phosphate | | Pharma S.a.u. | |
| | Water | — | — | 60 |

### Example 1

### Production of Microneedle Coated with Water-Soluble Steroid

Each of the coating liquids shown in Table 1 was coated onto a polyethylene terephthalate (PET) film so as to have a thickness of 150 μm, a needle of the above-described microneedle array was brought into contact with the coated surface so as to be vertical from the tip side, and the tip of the needle was allowed to reach the film surface. After the reach, the needle was held for around 5 seconds, and then the needle of the microneedle array was removed from the coated surface. After that, the coating liquid was fixed (coated) by drying for 12 hours to 24 hours with the needle facing in an upward direction.

### Test Example 1

### Measurement Test of Drug Concentration in Tissue Near Meibomian Glands by Perforation of Microneedle Coated with Water-Soluble Steroid, by Using Rabbit Palpebral Conjunctiva

### <Test Method>

Anesthesia was performed on a rabbit (Slc: JW/CSK, Japanese white color species, Japan SLC, Inc.) by inhalation of isoflurane, and then the eyelid was inverted to expose the palpebral conjunctiva. In the exposed palpebral conjunctiva, the microneedle was perforated by hand for 1 second or 5 seconds with the use of the water-soluble steroid-coated microneedle array prepared in Production Example 1. In this regard, in order to suppress the bleeding due to the microneedle perforation, 50 μL of naphazoline hydrochloride (NPZ) being vasoconstrictor eye drops was instilled into the eye by using a pipetter each of 10 minutes before and 5 minutes before the perforation.

Immediately after the perforation, the perforation portion was cleaned with gauze, and then the surrounding tissue including a meibomian gland (hereinafter, also expressed as meibomian gland surrounding tissues) was removed. The drug concentration in the removed surrounding tissue including a meibomian gland was measured by the procedures described below.

### <<Measurement Method for Drug Concentration in Meibomian Gland Surrounding Tissues>>

The meibomian gland surrounding tissues that have been excised were cut into small pieces with scissors, and then the obtained small pieces were sent to a centrifuge tube, 1 mL of a solution of water/acetonitrile/methanol=54/35/11 was added into the centrifuge tube to immerse the small pieces, and the centrifuge tube was left to stand overnight in a refrigerator. Centrifugation was performed at 10,000 rpm for 10 minutes by using a centrifuge, and 0.8 mL of the supernatant was transferred to a separate test tube. The solvent contained in the supernatant was removed by blowing nitrogen gas, and the resultant mixture was dried and solidified, and then the dried and solidified mixture was redissolved by adding 0.5 mL of a solution of water/acetonitrile/methanol=54/35/11. Centrifugation was performed at 10,000 rpm for 10 minutes by using a centrifuge, 0.4 mL of the supernatant was filtered, and the drug concentration in the tissue was measured by a measurement method using HPLC analysis under the following conditions.

The utilization rate of the drug was calculated from the result of the drug concentration in the tissue and the concentration of the drug coated on the microneedle (before use).

In this regard, the concentration of the drug coated on the microneedle was measured by the following procedures.

### <<Measurement Method for Concentration of Drug Coated on Microneedle>>

A microneedle before use (perforation) was placed in a centrifuge tube in which 1 mL of Milli-Q water had been placed, and it was confirmed that the entire needle was immersed in the Milli-Q water. A lid of the tube was closed, and the tube was left to stand overnight in a refrigerator. The extract was recovered from the centrifuge tube, filtered, and the concentration of the drug coated on the microneedle was measured before perforation by a measurement method using HPLC analysis under the following conditions.

### <<HPLC>>

Device: LC-2010HT (Shimadzu Corporation)

Column: Kinetex 5 C8 100 A, 5 μm, 4.6 mm×250 mm (SHIMADZU GLC Ltd.)

Column temperature: 40° C.

Injection volume: 50 μL

Flow rate: 0.65 mL/min

Detection wavelength: 254 nm

Mobile phase: 0.1% phosphate buffer solution/acetonitrile/methanol=54/35/11

The obtained results are shown in Tables 2 and 3.

TABLE 2
Drug concentration in meibomian gland surrounding tissues

Drug concentration (μg/g)
Coating microneedle

| Administration time | | Prescription A | Prescription B |
|---|---|---|---|
| 1 | second | 4.7 ± 4.3 | 4.0 ± 3.6 |
| 5 | seconds | 15.7 ± 8.8 | 15.4 ± 9.7 |

* The number of tests: coating microneedle N = 3
Average value: ±S.D.

TABLE 3
Drug utilization rate in meibomian gland surrounding tissues

Utilization rate (%)
Coating microneedle

| Administration time | | Prescription A | Prescription B |
|---|---|---|---|
| 1 | second | 5.0 ± 4.6 | 5.1 ± 4.6 |
| 5 | seconds | 16.9 ± 9.5 | 19.9 ± 12.6 |

* The number of tests: coating microneedle N = 3
Average value: ±S.D.

In a case where a hydrophobic steroid is administered to the upper eyelid skin by the application of ointment, it is known that the concentration (Cmax) in tissue of around 2.1 (±2.5) μg/g is observed in 15 minutes after administration in the palpebral conjunctiva including a meibomian gland (Lotemax (registered trademark) Ointment 0.5% Product Monograph, 2004).

In the present invention, by administering a water-soluble steroid in a form of a microneedle to which drug-containing coating had been performed, as compared with the drug concentration in the administration of ointment for 15 minutes, the drug concentration in the meibomian gland surrounding tissues was observed as follows: the drug concentration was around twice in only 1 second after the administration, and around 7 times in 5 seconds after the administration (see Table 2). That is, according to the present invention, the drug concentration in the meibomian gland surrounding tissues can be increased up to around 15 μg/g in only 5 seconds after the administration. That is, 0.050 μg/g or more of a steroid drug, which is considered to be a sufficient amount, can reach the affected part in a short time.

Further, according to the present invention, it was able to be confirmed that by the perforation for only 5 seconds, the utilization rate of the drug was increased to around 17% to 20% on average, and both of the administration rate and the administration efficiency were dominant.

As described above, according to the present invention, as compared with the conventional treatment method, the sufficient administration concentration is realized in a surrounding tissue including a meibomian gland, which is an affected part, in a short time, and the long-term use of a steroid can be avoided. As a result, there are no increased intraocular pressure, cataract, corneal epithelium disorder, delay of corneal wound healing, and infections, which are induced by long-term use of a conventionally used ophthalmic steroid such as steroid eye drops, and ophthalmic ointment, and also there are no other serious side effects such as transient ocular discomfort, and steroid-induced calcification. In addition, because of a short-time treatment, the uncomfortable feeling forced by the dosage form of a patch form is reduced, and therefore, it is conceivable that the present invention is a transdermal absorption delivery system that can contribute to a treatment method with less burden on a patient.

1. A method for administering a drug for treatment of eyelid disease or palpebral conjunctiva disease, comprising
a step of conducting a microneedle perforation by using a microneedle device into a tissue of the eyelid including a meibomian gland of a patient that has been infected with or that may be infected with the disease,
wherein drug-containing coating is applied to at least a part of a surface of the microneedle, and
the drug is a water-soluble drug.

2. The method according to claim 1, wherein the drug is at least a water-soluble steroid having an octanol/water distribution coefficient (log D) selectable in a range from −5 to 0.

3. The method according to claim 1, wherein the drug is at least one water-soluble steroid selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.

4. The method according to claim 1, wherein the drug-containing coating contains at least one kind of water-soluble polymer selectable from the group consisting of polyhydroxymethyl cellulose, polyhydroxypropyl methyl cellulose, polymethyl cellulose, hydroxyethyl cellulose, hydroxypropyl cellulose, hydroxypropyl methyl cellulose, carboxymethyl cellulose, carboxypropyl cellulose, dextran, polyethylene glycol, polyvinyl alcohol, polyvinyl pyrrolidone, pullulan, chondroitin sulfate or an ester salt thereof, and hyaluronic acid or a salt thereof.

5. The method according to claim 1, wherein the step of conducting the microneedle perforation is a step of conducting a microneedle perforation into a surface of the eyelid skin or a surface of the palpebral conjunctiva.

6. The method according to claim 1, wherein the step of conducting the microneedle perforation is a step of conducting a microneedle perforation for 1 second to 10 seconds.

7. The method according to claim 1, further comprising a step of applying a vasoconstrictor to the tissue of the eyelid.

8. The method according to claim 1, wherein the eyelid disease or the palpebral conjunctiva disease is at least one disease selectable from the group consisting of chalazion, blepharitis, meibomian gland dysfunction, allergic conjunctivitis, and vernal keratoconjunctivitis.

9. A microneedle device for administering a drug for treatment of eyelid disease or palpebral conjunctiva disease to a tissue of the eyelid including a meibomian gland, comprising
a microneedle,
wherein coating containing the drug is applied to at least a part of a surface of the microneedle, and
the drug is a water-soluble drug.

10. The microneedle device according to claim 9, wherein the microneedle device is a device for administering the drug through the eyelid skin or the palpebral conjunctiva.

11. A microneedle device for administering a drug for treatment of eyelid disease or palpebral conjunctiva disease to a tissue of the eyelid including a meibomian gland through the eyelid skin or the palpebral conjunctiva, comprising
a microneedle,
wherein coating containing the drug is applied to at least a part of a surface of the microneedle, and
the drug is a water-soluble drug.

12. The microneedle device according to claim 9, wherein the drug is at least a water-soluble steroid having an octanol/water distribution coefficient (log D) selectable in a range from −5 to 0.

13. The microneedle device according to claim 9, wherein the drug is at least one water-soluble steroid selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.

14. The microneedle device according to claim 9, wherein the coating contains at least one kind of water-soluble polymer selectable from the group consisting of polyhydroxymethyl cellulose, polyhydroxypropyl methyl cellulose, polymethyl cellulose, hydroxyethyl cellulose, hydroxypropyl cellulose, hydroxypropyl methyl cellulose, carboxymethyl cellulose, carboxypropyl cellulose, dextran, polyethylene glycol, polyvinyl alcohol, polyvinyl pyrrolidone, pullulan, chondroitin sulfate or an ester salt thereof, and hyaluronic acid or a salt thereof.

15. The microneedle device according to claim 9, wherein the eyelid disease or the palpebral conjunctiva disease is at least one disease selectable from the group consisting of chalazion, blepharitis, meibomian gland dysfunction, allergic conjunctivitis, and vernal keratoconjunctivitis.

16. The microneedle device according to claim 9, wherein the microneedle device is used for treatment of eye disease due to dysfunction of a tissue of the eyelid.

17. The microneedle device according to claim 9, wherein the microneedle device is used for treatment of eye disease.

18. The microneedle device according to claim 9, further comprising a microneedle array provided with the microneedle, wherein the microneedle array has an area of 0.01 cm² or more and 1.20 cm² or less.

19. A method for treatment of eyelid disease or palpebral conjunctiva disease, comprising a step of making a microneedle in the microneedle device according to claim 9, perforated into the eyelid skin or a surface of the palpebral conjunctiva of a patient that has been infected with or that may be infected with disease.

20. The method according to claim 19, wherein the step of conducting the microneedle perforation is a step of conducting a microneedle perforation for 1 second to 10 seconds.

21. The method according to claim 19, further comprising a step of applying a vasoconstrictor to a tissue of the eyelid.

22. A set for treatment of eyelid disease or palpebral conjunctiva disease, comprising: the microneedle device according to claim 9, and a drug product containing one or both of a support base for eyelid skin and a vasoconstrictor.
